(19) United States
(12) Patent Application Publication
(10) Pub. No.: US 2017/0224900 A1
(43) Pub. Date: Aug. 10, 2017

(54) Device and Method for Gaining Access into a Body Cavity

(71) Applicants: Judson E. Threlkeld, Camas, WA (US); Joe Hare, Seattle, WA (US); Robert L. Parton, Arcata, CA (US)

(72) Inventors: Judson E. Threlkeld, Camas, WA (US); Joe Hare, Seattle, WA (US); Robert L. Parton, Arcata, CA (US)

(21) Appl. No.: 15/019,136

(22) Filed: Feb. 9, 2016

(51) Int. Cl. A61M 1/36; A61B 17/12; A61M 25/06

(52) U.S. Cl. CPC A61M 1/3661; A61M 25/065; A61B 17/1204; A61B 17/12109; A61M 25/09

(57) ABSTRACT

A device and methodology for gaining access into a body cavity of a patient is described and which includes an elongated sheath; an elongated dilator which is telescopingly received in the sheath, and wherein the elongated dilator has a distal end which is received within a body cavity to be accessed, a selectively inflatable occlusion balloon mounted on the dilator, and first and second sources of pressurized fluid which is coupled in fluid flowing relation relative to the dilator, and which further is effective, on the one hand, for inflating the occlusion balloon, and secondly for delivery into the body cavity of the patient based on the desires of a clinician.

### TECHNICAL FIELD

The present invention relates to a device and method for gaining access into a body cavity, and more specifically to a device, which when deployed, allows a clinician to gain access to a body cavity such as a blood vessel, or fistula, and further permits the clinician to perform various medical procedures in a manner not possible, heretofore.

### BACKGROUND OF THE INVENTION

The establishment of vascular hemodialysis access for patients having renal failure has created many challenges for treating physicians. In this regard, vascular access is a principal cause of morbidity, and cost, in hemodialysis patients. Vascular access costs for hemodialysis are quite high. Complications associated with vascular access has been estimated to be in the range of 10% to 15% of all inpatient hospital admissions experienced by hemodialysis patients. Heretofore, arteriovenous fistulas (AVF's) have been the preferred vascular access for hemodialysis. It has long been the view of many physicians that mature AVF's require fewer interventions to maintain their long term usefulness than do arteriovenous grafts, for example.

One of the chief disadvantages of AVF's has included the inability of such AVF's to be used in acute settings because the time needed for full maturation of an AVF typically takes about three to four months. Fistula maturation is indicated by dilatation, augmented blood flow, and wall thickening which is sufficient to support hemodialysis. As should be understood, maturation probability is often difficult to predict. It is reported, however, that 60% of AVF's fail to mature adequately. Indeed, it is reported that approximately 40% of patients who present end-stage renal disease require dialysis at the time of initial clinical presentation. This explains, in part, why synthetic vascular access (SVA) devices have become one of the most attractive treatment options for many dialysis patients because it is typically difficult or impossible to establish a mature AVF prior to initiation of chronic dialysis treatments for these patients.

A synthetic vascular access (SVA) is frequently placed in patients who fail AVF placement, or in whom the availability of a vascular access is desired because they can be used one or two weeks after placement. It is reported that a synthetic vascular access typically has a higher failure/complication rate than those associated with AVF's due primarily to the development of stenosis at the venous anastomosis resulting from initial hyperplasia. To address the failure/complications associated with SVA's various procedures have been developed. However, these procedures have proved less than satisfactory for a number of different reasons, and clinicians have sought after other means of gaining access to a body cavity such as a blood vessel or fistula (AVF or SVA) and which does not employ the multi-step processes which have been utilized, heretofore.

A device or methodology for gaining access to a body cavity which avoids the detriments associated with the prior art devices and practices utilized, heretofore, is the subject matter of the present patent application.

### SUMMARY OF THE INVENTION

A first aspect of the present invention relates to a device for gaining access into a body cavity and which includes an elongated sheath having opposite ends, and which further defines a longitudinally oriented passageway which extends between the opposite ends; an elongated dilator having a main body with opposite ends, and which is telescopingly received within the longitudinally extending passageway which is defined by the sheath, and wherein the dilator defines a longitudinally oriented lumen which extends between the opposite ends thereof, and further has a first and second passageway which is formed in the main body of the elongated dilator; a selectively inflatable occlusion balloon mounted on the dilator, and which is coupled in fluid flowing relation relative to the first passageway which is defined by the elongated dilator; a first source of pressurized fluid which is coupled in fluid flowing relation relative to the first passageway; and a second source of pressurized fluid which is coupled in fluid flowing relation relative to the second passageway.

Still another aspect of the present invention relates to a device for gaining access into a body cavity, and which includes an elongated sheath having opposite first, and second ends, an exterior facing surface, and a longitudinally extending passageway which extends between the opposite first and second ends, and wherein the longitudinally extending passageway has a predetermined inside diametral dimension; an elongated dilator having a main body which has an outside diametral dimension which is less than the inside diametral dimension of the longitudinally extending passageway which is defined by the elongated sheath, and wherein the main body of the elongated dilator further has opposite, first and second ends, and wherein the second end of the dilator is received within a body cavity of a patient, and wherein the main body of the elongated dilator has a longitudinally oriented lumen which extends between the opposite first and second ends of the main body, and wherein the elongated dilator is further telescopingly received within the longitudinally extending passageway of the sheath, and is further reciprocally and longitudinally moveable relative to the elongated sheath, and wherein the main body of the elongated dilator further has an outside facing surface, and wherein a first and second passageway is individually formed in the main body of the elongated dilator, and which further, respectively, extend from the first end thereof, and in the direction of the second end of the main body, and wherein each of the first and second passageways have a proximal, and a distal end; a selectively inflatable occlusion balloon mounted on the dilator at a location which is intermediate the first and second ends of the main body thereof, and wherein the dilator balloon is coupled in selective, fluid flowing relation relative to the first passageway which is formed in the main body of the elongated dilator, and wherein the occlusion balloon can be inflated when the elongated dilator is longitudinally advanced to a position where the occlusion balloon is located longitudinally, outwardly, relative to the second end of the elongated sheath, and wherein the second passageway which is formed in the main body of the dilator, and further defined, at least in part, by the exterior facing surface thereof, extends from the first end of the dilator and in the direction of the second end of the dilator, and wherein the distal end of the second passageway terminates at a location which is adjacent to, but short of the selectively inflatable occlusion balloon, and wherein the main body of the elongated dilator can be longitudinally advanced relative to the sheath so as to position both the occlusion balloon, and the distal end of the second passageway which is formed in the main body of the elongated dilator, at a location which is longitudinally, outwardly, relative to the second end of the sheath; a first source of a pressurized fluid which is coupled in selective, fluid delivering relation relative to the proximal end of the first passageway, and which is defined by the elongated dilator, and wherein the first source of pressurized fluid, when delivered to the first passageway is effective in inflating the occlusion balloon; and a second source of a pressurized fluid which is coupled in selective, fluid delivering relation relative to the proximal end of the second passageway of the elongated dilator, and which further, when delivered to the second passageway, travels to the distal end thereof, and then exists the elongated dilator in a direction which is oriented laterally, outwardly relative thereto.

Still another aspect of the present invention relates to a method for gaining access to a body cavity which includes selecting a body cavity for access; inserting a needle, at least in part, into the selected body cavity, and wherein the needle defines a longitudinally oriented passageway extending therethrough; inserting a wire into the longitudinally oriented passageway, and orienting the wire in a given location within the selected body cavity; removing the needle from the body cavity while maintaining the wire within the selected body cavity; providing a sheath having a main body which defines a longitudinally extending passageway having a predetermined inside diametral dimension, and extending the wire through the longitudinally extending passageway; movably advancing the sheath along the wire, and inserting at least a portion of the sheath into the selected body cavity; removing the wire from the selected body cavity while maintaining at least a portion of the sheath within the selected body cavity; providing a dilator with a main body which defines a longitudinally extending lumen, and first and second passageways, and telescopingly orienting the main body of the dilator within the longitudinally extending passageway which is defined by the sheath, and inserting, at least in part, a portion of the main body of the dilator within the selected body cavity; mounting a selectively inflatable occlusion balloon in a given location on the main body of the dilator, and coupling the occlusion balloon in fluid flowing relation relative to the first passageway as defined by the dilator; selectively coupling a first source of a pressurized fluid to the first passageway defined by the dilator, and delivering the first source of pressurized fluid to the first passageway so as to effect an inflation of the occlusion balloon within the selected body cavity; and selectively coupling a second source of a pressurized fluid to the second passageway, and delivering the second source of the pressurized fluid by way of the second passageway to the selected body cavity.

Yet still another aspect of the present invention relates to a method for gaining access to a body cavity which includes inserting an elongated needle, at least in part, into a body cavity of a patient, and wherein the elongated needle has a proximal and distal end, and further has a longitudinally oriented passageway which extends between the proximal and distal ends thereof; inserting a wire into the longitudinally extending passageway of the elongated needle, and advancing the inserted wire through the needle, and further orienting a portion of the wire in the body cavity which is being accessed; after the step of inserting a portion of the wire into the body cavity which is being accessed, removing the needle from the body cavity by passing the needle over the wire, and simultaneously leaving the portion of the wire previously inserted in the body cavity which is being accessed, within the body cavity; providing a sheath having opposite first, and second ends, and a longitudinally extending passageway which extends between the opposite first and second ends, and wherein the longitudinally extending passageway of the sheath has a predetermined inside diametral dimension, and wherein the method further includes passing the wire, at least in part, through the longitudinally extending passageway of the sheath, and further moveably advancing the sheath along the wire, and inserting the second end of the sheath within the body cavity which is being accessed; after the step of providing the sheath, the method includes another step of removing the wire from the body cavity which is being accessed by forcibly withdrawing the wire through the longitudinally extending passageway which is defined by the sheath; after the step of removing the wire from the body cavity, the method further includes a step of providing an elongated dilator having a main body which has an outside diametral dimension which is less than the inside diametral dimension of the longitudinally extending passageway which is defined by the elongated sheath, and wherein the main body of the elongated dilator further has opposite, first and second ends, and a longitudinally oriented lumen which extends between the opposite first and second ends of the main body, and wherein the main body of the elongated dilator further has an outside facing surface, and wherein a first and second passageway is individually formed in the main body of the elongated dilator, and wherein each of the first and second passageways have a proximal, and a distal end, and which further, respectively, extend from the first end thereof, and in the direction of the second end of the main body, and wherein the method further includes the step of telescopingly inserting the main body of the dilator within the longitudinally extending passageway which is defined by the sheath, and longitudinally advancing the dilator along the longitudinally extending passageway which is defined by the sheath, and orienting the second end of the dilator at a location which is longitudinally, outwardly relative to the second end of the sheath, and within the body cavity being accessed, and wherein the dilator is selectively, reciprocally, and longitudinally moveable relative to the second end of the elongated sheath; providing a selectively inflatable occlusion balloon, and mounting the occlusion balloon on the dilator at a location which is intermediate the first and second ends of the main body thereof, and further coupling the dilator balloon in selective, fluid flowing relation relative to the distal end of the first passageway which is formed in the main body of the elongated dilator, and wherein the method further includes longitudinally advancing the occlusion balloon relative to the dilator to a position where the occlusion balloon is located longitudinally, outwardly, relative to the second end of the elongated sheath, and wherein the second passageway which is formed in the outside facing surface of the main body of the dilator extends from the first end thereof, and the distal end of the second passageway terminates at a location which is adjacent to, but short of the selectively inflatable occlusion balloon, and wherein the main body of the elongated dilator is selectively, longitudinally advanced relative to the sheath so as to position both the occlusion balloon, and the distal end of the second passageway, and which is formed in the main body of the elongated dilator, at a location which is longitudinally, outwardly relative to the second end of the sheath; providing a first source of a pressurized fluid and coupling the first source of pressurized fluid in selective, fluid delivering relation relative to the proximal end of the first passageway of the elongated dilator, and delivering the first source of the pressurized fluid to the proximal end of the first passageway so as to effect an inflation of the occlusion balloon within the body cavity which is being accessed; and providing a second source of a pressurized fluid, and coupling the second source of the pressurized fluid in selective, fluid delivering relation relative to the proximal end of the second passageway of the elongated dilator, and wherein the method further includes the step of delivering the second source of pressurized fluid to the second passageway, for movement therealong to the distal end of the second passageway, and further depositing the second source of the pressurized fluid within the body cavity being accessed, and wherein the second source of pressurized fluid exists the elongated dilator in a direction which is oriented laterally, outwardly relative thereto, and into the body cavity being accessed.

These and other aspects of the present invention will be discussed in greater detail hereinafter.

### BRIEF DESCRIPTION OF THE DRAWINGS

Preferred embodiments of the invention are described below with reference to the following accompanied drawings.

FIG. 1 is a perspective, exploded, side elevation view of a disassembled device for insertion into a body cavity in accordance with the teachings of the present invention.

FIG. 2 is a fragmentary, perspective, greatly enlarged, side elevation view of a portion of the device as seen in FIG. 1.

FIG. 3 is a transverse, vertical sectional view of a portion of the device, and which is taken from a position along line 3-3 of FIG. 2.

FIG. 4 is a fragmentary, greatly enlarged, proximal, longitudinal end view of the portion of the device as seen in FIG. 2.

FIG. 5 is a fragmentary, side elevation view of an elongated sheath which forms a feature of the present invention,

FIG. 6 is a greatly enlarged, proximal, longitudinal end view of the sheath as seen in FIG. 5, and which forms a feature of the present invention.

FIG. 7 is a graphical depiction of a step in the methodology of the present invention.

FIG. 8 is a graphical depiction of another step in the methodology of the present invention.

FIG. 9 is a graphical depiction of still another step in the methodology of the present invention,

FIG. 10 is a graphical depiction of still another step in the methodology of the present invention,

FIG. 11 is a greatly enlarged, graphical depiction of the device of the present invention implementing yet another series of steps in the methodology thereof.

### DETAILED DESCRIPTION OF THE PREFERRED EMBODIMENTS

This disclosure of the invention is submitted in furtherance of the constitutional purposes of the U.S. Patent Laws “to promote the progress of science and useful arts” (Article 1, Section 8).

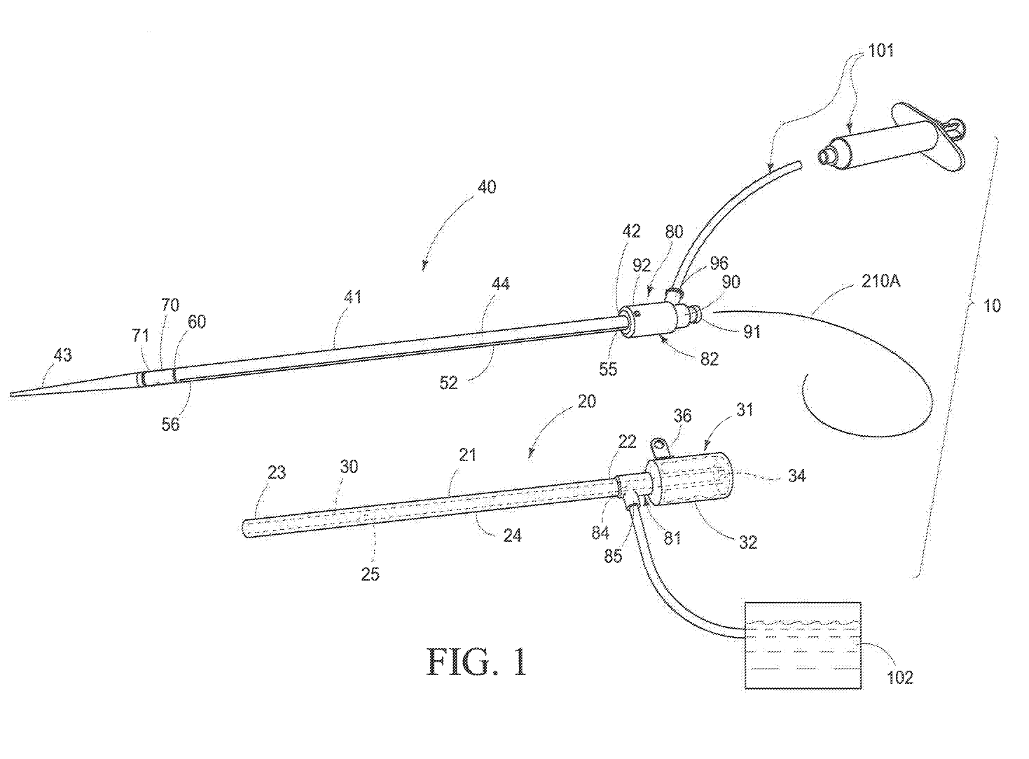

The present invention is generally indicated by the numeral 10 in FIG. 1 and following.

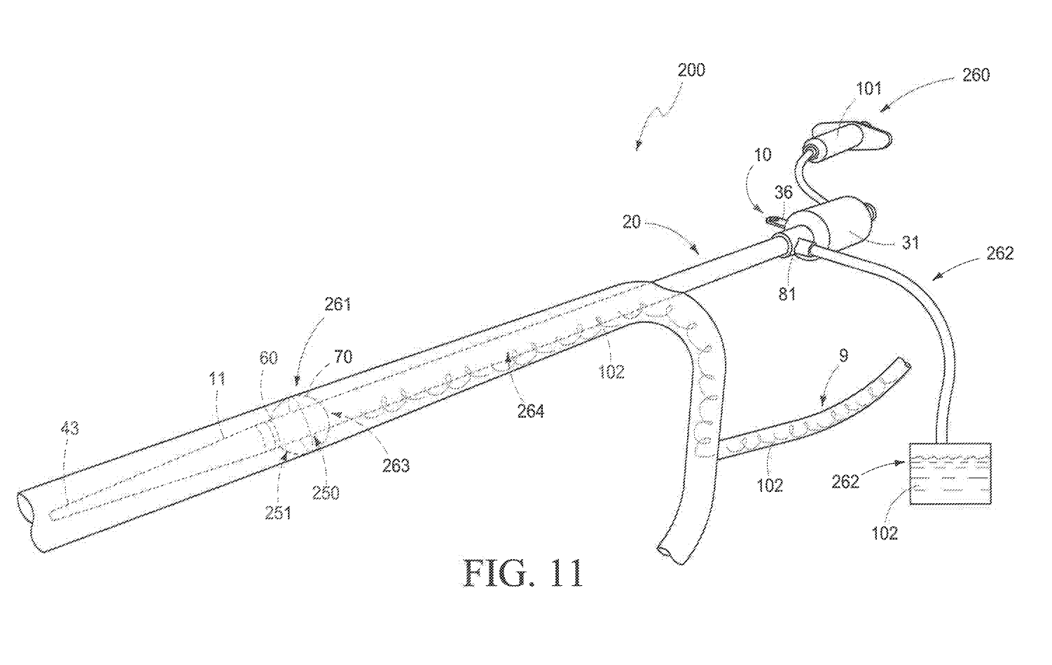

As background, the present invention allows for more efficient access to a body cavity such as a blood vessel, surgically created fistula (AVF) or a Synthetic Vascular Access (SVA) which is implanted in a patient undergoing hemodialysis. As should be appreciated, vascular access is a hemodialysis patient's lifeline. Vascular access makes life-saving hemodialysis treatments possible. Hemodialysis is a treatment for kidney failure that uses a machine (not shown) to send the patient's blood through a filter called a dialyzer, outside the body. The vascular access 9 (FIG. 11) is typically surgically created in a vein, and which is used to remove, and then return blood during the hemodialysis treatment. In this regard, the blood of the patient goes through a needle, a few ounces at a time. The blood then travels through a tube that takes it to the dialyzer. Inside the dialyzer (not shown) the blood flows through thin fibers that filter out waste and extra fluid. The dialysis machine then returns the filtered blood to the body of the patient through a different tube. A vascular access lets a large amount of the patient's blood flow continuously through the hemodialysis machine to the filter so that as much blood, as possible, is filtered per treatment. A pint of blood typically flows through the machine approximately every minute. As earlier discussed, a surgically created vascular access (AVF) should be in place weeks or months before the first hemodialysis treatment to ensure success. Ideally, many physicians prefer the formation of an arteriovenous fistula (AVF) to permit access to the patient's blood during dialysis. AVF's have been preferred, over time, because venous stenosis, and infections occur with much less frequency than what is seen with other procedures, such as grafts. It has been reported that thrombosis rates for an AVF is one-sixth that for a graft, while the infection rate is approximately one-tenth. For an AVF to be successful, it is reported that several requirements must be met. Firstly, the blood flow provided by the AVF must be adequate to support dialysis. Secondly, the AVF must maturate adequately to allow for repetitive cannulation. Thirdly, and ideally, the AVF must be located within about one centimeter of the skin surface for easy access.

Further, a relatively straight segment of the AVF must be available for cannulation. Finally, an anatomical location in the patient's body must be selected which is accessible by a physician, or other health care worker, during the dialysis process. Typically this anatomical location must be accessible while the patient is oriented in a sitting position.

As noted earlier in this patent application, a large portion of end-stage renal disease patients present symptoms that require immediate dialysis at a time when the creation of an AVE, and its full maturation is not deemed possible. Because a mature AVE cannot be created, synthetic vascular access (SVA) have been created so as to allow end-stage renal disease patients (ESRD) and others to have life-saving hemodialysis performed. These SVA's can be surgically implanted and then can be used in a very short period of time and often months prior to a properly created AVE.

As should be appreciated, the various types of vascular access for end-stage renal failure patients can cause problems that require further treatment or even surgery. The most common problems include access infection, low blood flow due to blood clotting in the access, and other issues. Generally speaking, infection and low blood flow happen less frequently in properly formed AVF's than in an AV graft, for example, and/or the use of venous catheters. However, problems can still exist in synthetic vascular access (SVA) structures, as well. For example, arteriovenous grafts more often develop low blood flow, and indications of clotting or narrowing of the access. An AV graft may then require angioplasty which is a procedure to widen the narrow part. Another option involves surgery on the arteriovenous graft, to replace the narrow part. Venous catheters, for example, are most likely to cause infection and clotting problems. The present device 10 (FIG. 1) and methodology, as described hereinafter, may be employed to address many of the aforementioned problems associated with access to a body cavity 11 such as a blood vessel or other fistula, whether naturally created, or synthetic, as will be discussed, hereinafter. In the specification which follows, the body cavity is generally indicated by the numeral 11, and may include a blood vessel; a surgically formed fistula, or synthetic vascular access device which are placed under the skin of the patient and which allow a clinician to gain access to the blood flow of the patient so that it may be diverted to a hemodialysis machine (not shown) for further treatment, and then subsequently returned to the patient's body.

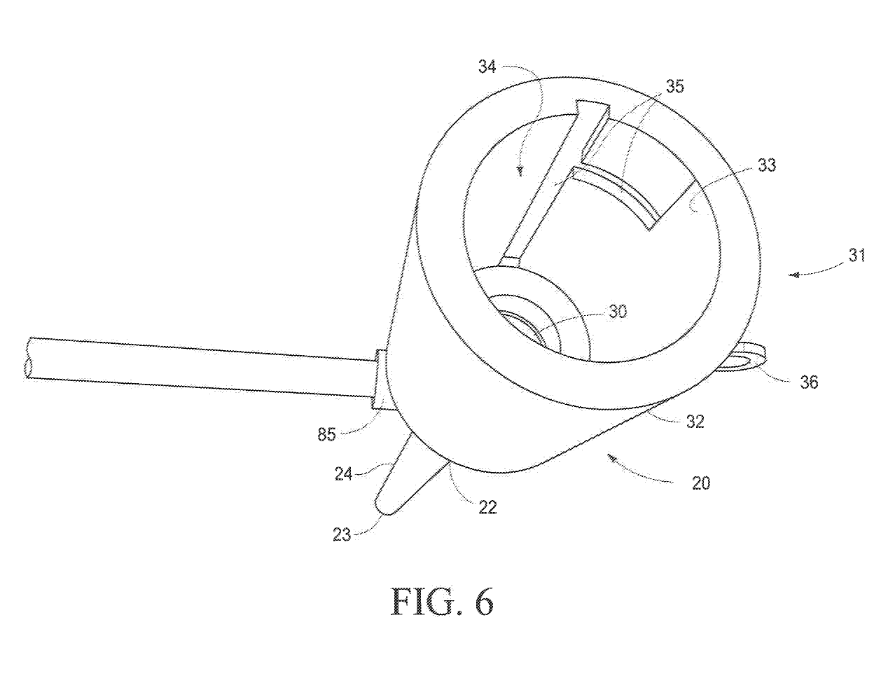

Referring now to the drawings which are provided, the device 10, of the present invention includes, as a first feature, an elongated, somewhat flexible sheath which is generally indicated by the numeral 20. The sheath has an elongated main body 21 which has a first or proximal end 22, and a second, or distal end 23. The main body of the sheath 21 has an exterior facing surface 24, and an opposite, interior facing surface 25. The exterior facing surface 24 of the main body 21 is narrowly cylindrical in shape, and the second or distal end 23 is operable as will be described hereinafter, to be received, at least in part, within the body cavity 11 using the methodology as will be set forth later in this patent application. The interior facing surface 25 defines a longitudinally oriented passageway 30 which extends from the first or proximal end 22, to the second or distal end 23. The longitudinally oriented passageway 30 has a cross sectional dimension which is typically substantially uniform along its entire length. The sheath 20, at the first end 22 thereof includes an enlarged sheath hub 31. The sheath hub is defined by an outside facing surface 32, and an opposite, inside facing surface 33. The inside facing surface defines an internal cavity 34. As seen in FIG. 6, the inside facing surface 33 defines guidance channels 35 which facilitate the rotatable locking of the sheath 20 relative to a dilator, which will be discussed, below. Further a force engagement member 36 is mounted on, and extends radially, outwardly, relative to the outside surface 32 so as to allow a rotational force to be applied to the sheath hub 31 so as to effect a locking of sheath 20 to the dilator, which is discussed in the paragraph which follows.

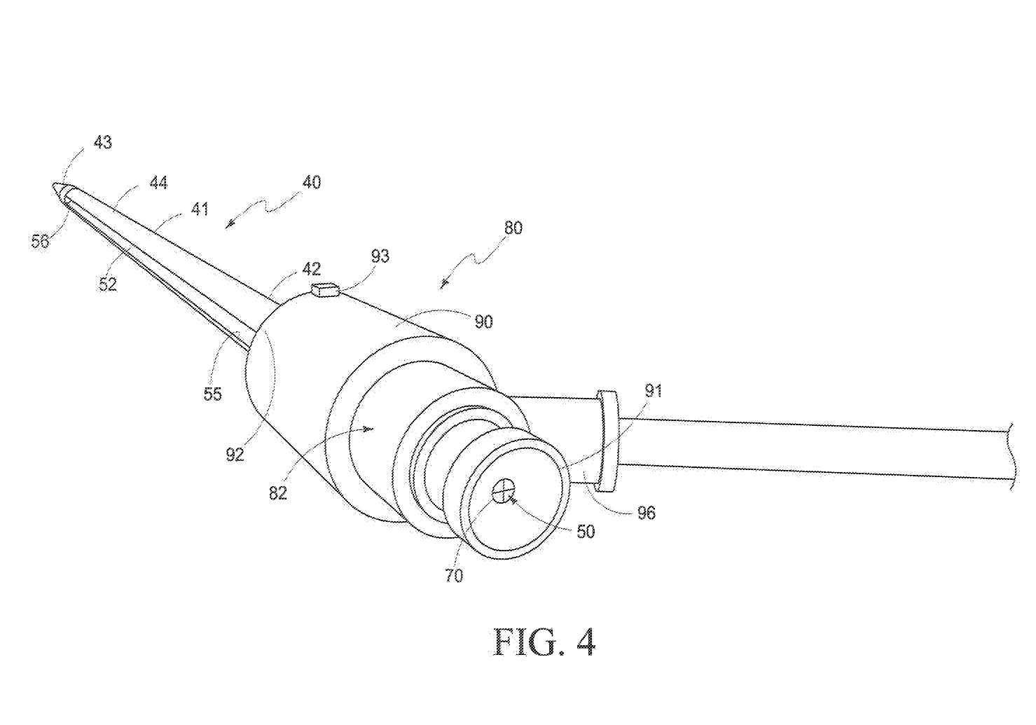

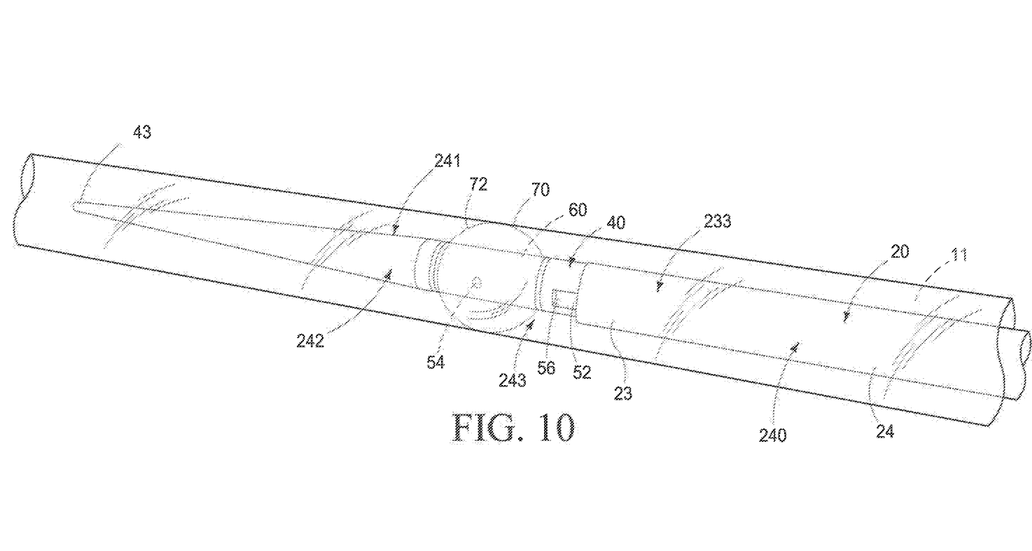

The sheath 20, as described above, is operable to cooperate with a dilator which is generally indicated by the numeral 40 in the drawings. The dilator has a narrowly elongated, and somewhat flexible main body which is generally indicated by the numeral 41. The dilator has a first or proximal end 42 which is located typically in spaced relation relative to the first end 22, of the sheath 20; and a second, opposite or distal end 43. When properly assembled the second end 43 is located in predetermined, spaced relation relative to the second end 23 of the sheath 20 (FIG. 10). The dilator has a cylindrically shaped exterior facing surface 44 which has an outside diametral dimension which is just slightly less than the inside diametral dimension of the longitudinally oriented passageway 30, and which is defined by the sheath 20. Therefore, the main body 41 of the dilator 40 can be telescopingly received, and is reciprocally, longitudinally moveable relative to the sheath 20 during operation. The operation of the device 10 will be discussed in greater detail later in this patent application. The interior facing surface 45 of the dilator 40 (FIG. 3) defines a longitudinally oriented lumen 50 (FIGS. 3 and 4) having a predetermined inside diametral dimension which can be somewhat variable when measured from the first end 42, and in the direction of the second distal end 43. As should be appreciated, the second or distal end 43 of the dilator is fabricated in a manner whereby the distal end 43 is a bit stiffer or less flexible than the remaining portion of the main body 41. The stiffer or less flexible second, or distal end 43 allows for easy insertion of the second or distal end 43 within the body cavity 11 as discussed earlier in this patent application.

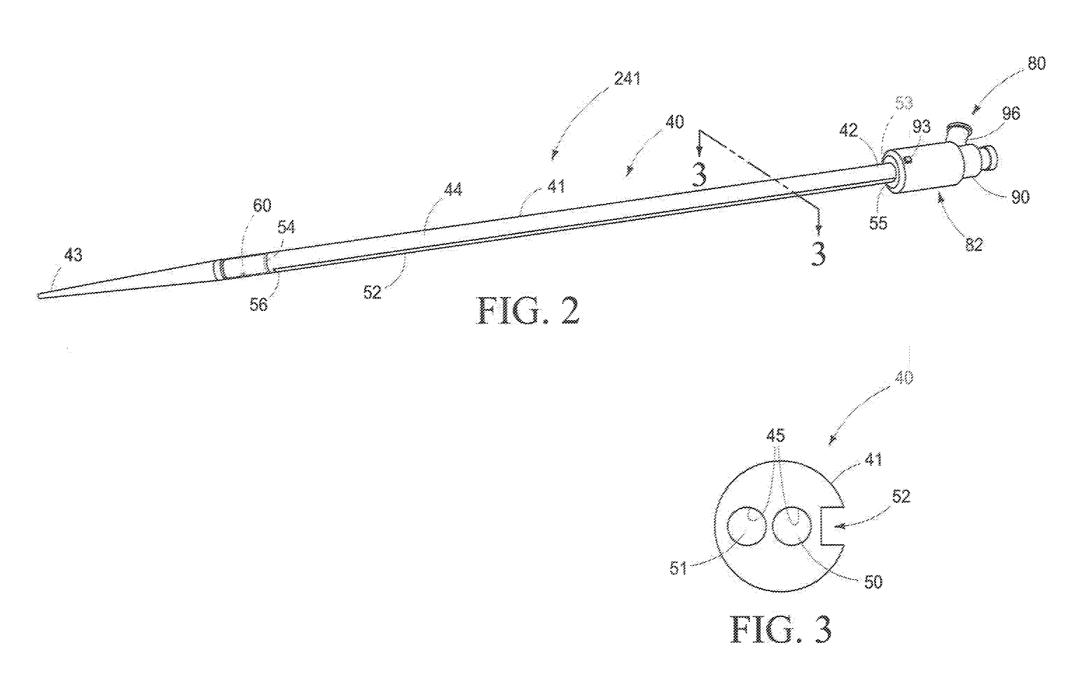

The dilator 40, as described above, and which is telescopingly received and is otherwise longitudinally, and reciprocally moveable relative to the sheath 20 further defines first and second longitudinally extending fluid passageways which are generally indicated by the numeral 51 and 52, respectively (FIGS. 3 and 4). With respect to the first passageway 51 the first passageway includes a proximal end 53 which is located in closely adjacent, spaced relation relative to first end 42, and which further is accessible through the exterior facing surface 44. The first passageway 51 further has a distal end 54, and which is located in fluid delivering relation relative to a circumscribing recessed region, as will be described below, and which mounts or otherwise cooperates with an occlusion balloon. This will also be discussed in greater detail in the paragraphs which follow. Still further, the second passageway 52 has a proximal end 55 (FIG. 2), and a distal end 56. The distal or terminal end 56 of the second passageway 52 is located in a position just short of the recessed region which will be discussed, below, and which receives or cooperates with an occlusion balloon. The second passageway 52 is defined, in part, by the exterior facing surface 44, and appears fluted (FIGS. 2 and 3). The second passageway 52, as noted, is defined, in part, by the exterior facing surface 44, and is further defined, in part, by the interior facing surface 25, of the sheath 20, when the dilator 40 is properly assembled, and is received within the sheath 20 as earlier described (FIGS. 10 and 11).

As noted in the paragraph, above, a circumscribing recessed region 60 is defined by the exterior facing surface 44, of the dilator 40, and is further located in a position which is intermediate to the first and second ends 42 and 43, respectively. The recessed region 60 matingly cooperates with an occlusion balloon 70 of traditional design. The occlusion balloon is operable to move from a deflated orientation 71, and which allows the main body 41 of the dilator 40 to be inserted within the sheath 20, and be longitudinally advanced to a position where the occlusion balloon 70 is located longitudinally outwardly relative to the second or distal end 23 of the sheath 20, and further, when inflated by a source of fluid as will be discussed in greater detail, hereinafter, increases in size to an enlarged, inflated orientation 72. In the inflated orientation 72, the occlusion balloon 70 is operable to occlude at least in part, a body cavity 11 so as to allow for a medical procedure to take place and which addresses a problem confronting a patient (FIG. 10). The occlusion balloon 70 is coupled in fluid flowing relation relative to the distal end 54, of the first passageway 51. It should be understood that the first passageway 51 is defined within the main body 41, of the dilator 40, and further extends between the first end 42, and in the direction of the second end 43. As seen in the drawings, the distal end 54 of the first passageway 51 is coupled in fluid flowing relation to the occlusion balloon 70, and terminates within the circumscribing recessed region 60. As further seen in the drawings (FIG. 4), a flow control valve 73, of traditional design, is mounted on the second or distal end 42 of the dilator 40, and which allows a clinician access into the body cavity 11, but simultaneously prevents body fluid from escaping through the longitudinally oriented lumen 50 of the dilator 40 when the lumen 50 is not being occluded. The operation of this feature will become more apparent in the discussion which follows regarding the methodology of the invention.

A fluid coupling which is generally indicated by the numeral 80 is mounted on, and is otherwise made integral with the proximal end 42 of the dilator 40. The fluid coupling 80 has two component portions, a first portion 81, and a second portion 82. The first portion 81 is mounted on or matingly cooperates, with the sheath 20, and the dilator 40, at the first ends, thereof, in order to fluid flowingly couple the first or proximal end 42 of the dilator, and more specifically proximal end 55 of the second passageway 52, with a source of a fluid which is to be deposited within the body cavity 11 of the patient being treated. The fluid which is delivered constitutes well known substances, such as contrast, which may be used in connection with x-ray procedures, or may further include a flushing fluid selected by the physician. The first portion of the fluid coupling 81 has a main body 83 which has, or defines, a longitudinally extending passageway 84, and which has an inside diametral dimension which is just slightly greater than the outside diametral dimension of the sheath 20. Further the main body 81 includes a fluid flowing side arm 85 which extends generally radially outwardly therefrom, and which further couples the first portion 81 of the fluid coupling 80 in fluid flowing relation relative to a source of fluid 102 which may include a contrast or a flushing fluid selected by a physician and which is delivered by way of the side arm 85.

As can be seen from the drawings, the second portion 82 of the fluid coupling 80 has a main body 90 which has a first end 91, and an opposite second end 92. The main body 90 defines, in part a portion of the first passageway 51 (FIG. 3). The main body further has an outside diametral dimension which is just slightly less than the inside diametral dimension of the internal cavity 34 of the sheath hub 31. The main body 90 is coupled in fluid flowing relation relative to the proximal end 55 of the second passageway 52, and which is defined by the dilator 40. The main body 90 further includes a balloon inflation port 96 which extends laterally outwardly relative to the main body 90, and which further allows a first source of a pressurized fluid 101 to be coupled in fluid delivering relation relative to the first passageway 51. As seen in the drawings the first pressurized source of fluid 101 may be delivered by means of a syringe of traditional design. The first source of pressurized fluid 101 passes through the second portion 82 of the fluid coupling 80, and is delivered to the proximal end 53 of the first passageway 51. The pressurized fluid 101 then travels along the first passageway 51, and is delivered by way of the distal end 54 of the passageway 51 into the occlusion balloon 70 so as to inflate the occlusion balloon to the position 72 as seen in the drawings. The first pressurized fluid 101 could include either a gas and/or a liquid depending upon the needs of the clinician. It should be understood that a second pressurized source of fluid 102, is delivered to the fluid flowing side arm 85, and is allowed to pass along the second passageway 52, and be delivered to the distal or terminal end 56. The second source of fluid 102, as supplied, exits the terminal end 56, and is directed laterally or radially outwardly relative to the main body 41 of the dilator 40 (FIG. 11). The second source of fluid is delivered within the body cavity 11 of the patient (FIG. 11). The main body 90 further includes a coupling post or tab 93 (FIG. 4), and which is sized so as to mechanically cooperate with the guidance channels 35 and which are defined by the sheath hub 31. When the dilator 40 is properly received within the internal cavity 34 of the sheath hub 31, the tab 93 allows the dilator to be properly oriented and locked into place so as to ensure the proper, orientation and fluid coupling of the first and second sources of fluid 101 and 102 with the respective passageway 51 and 52, respectively.

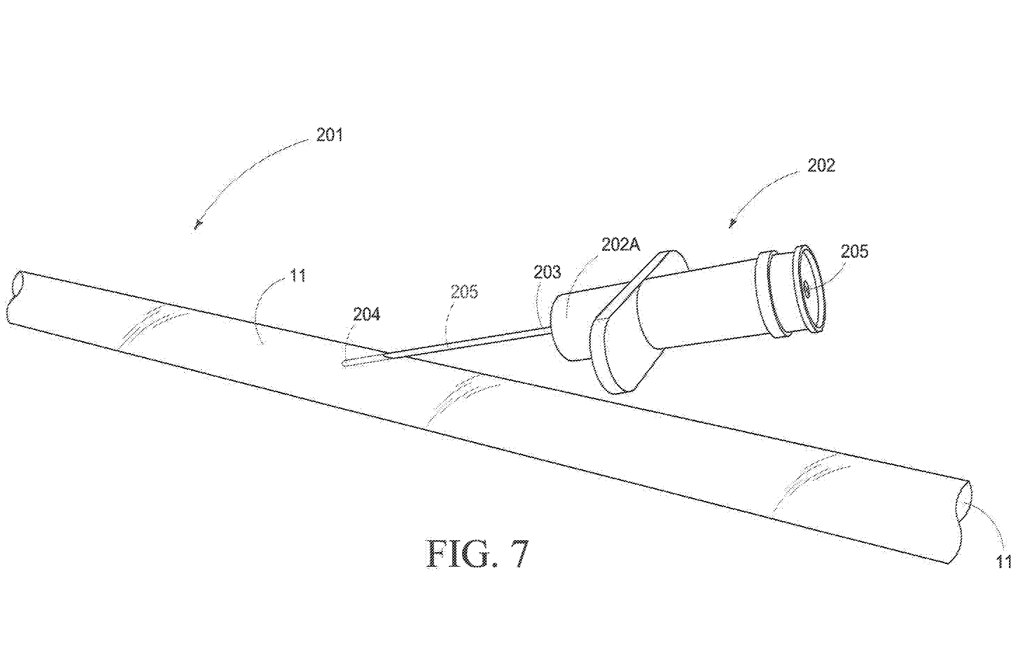

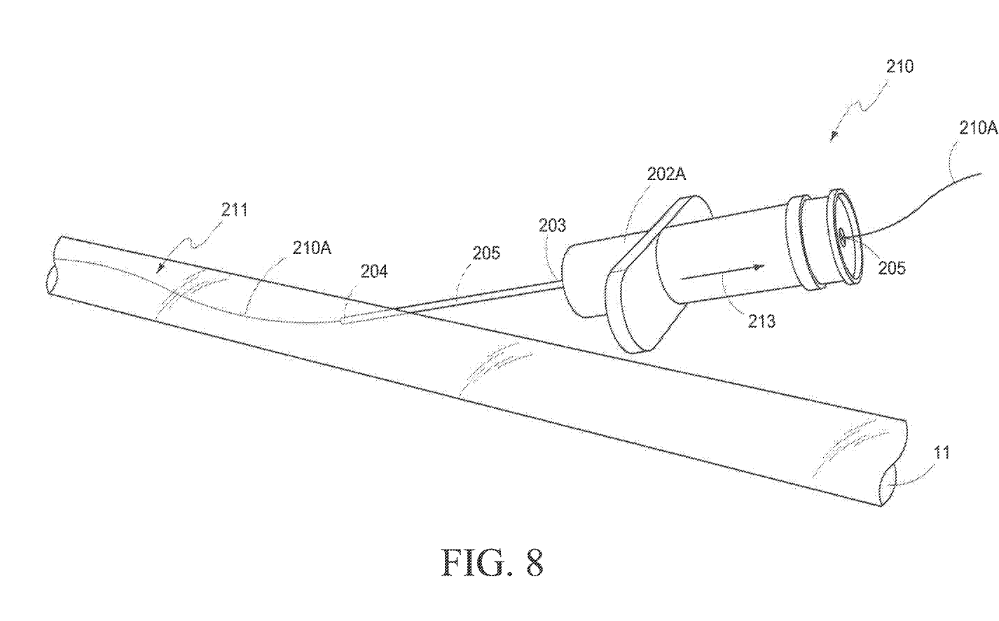

The device 10 of the present invention, and as shown in the drawings, can be utilized in a method for gaining access to the body cavity 11 of a patient and which includes the steps as identified, below (FIG. 11). This method for gaining access which is generally indicated by the numeral 200 includes, a first step 201 of selecting a body cavity 11 which needs access (FIG. 7). As earlier discussed, the body cavity 11 which is selected, or which is formed in a patient can typically be accessed while the patient is sitting in an upright position, although other locations can be chosen based upon the clinical judgment of the physician and needs of the patient. The methodology of the present invention has a second step 202 which includes inserting an elongated needle 202A, at least in part, into the body cavity 11 of a patient. In this step the elongated needle has a proximal end 203, and a distal end 204. The needle further has a longitudinally oriented passageway 205 which extends between the proximal and distal ends thereof 203 and 204, respectively. The third step 210 in the methodology for gaining access to a body cavity of a patient 200 includes inserting a wire 210A through the needle 202; and a fourth step 211 (FIG. 8) of further orienting a portion of the wire 210A in the body cavity 11 which is being accessed. After the step 210 of inserting the wire into the body cavity 11 which is being accessed, the fifth step 213 of the present methodology includes removing the needle from the body cavity 11 by passing the needle over the wire, and simultaneously leaving a portion of the wire previously inserted into the body cavity 11 which is being accessed, within the body cavity 11.

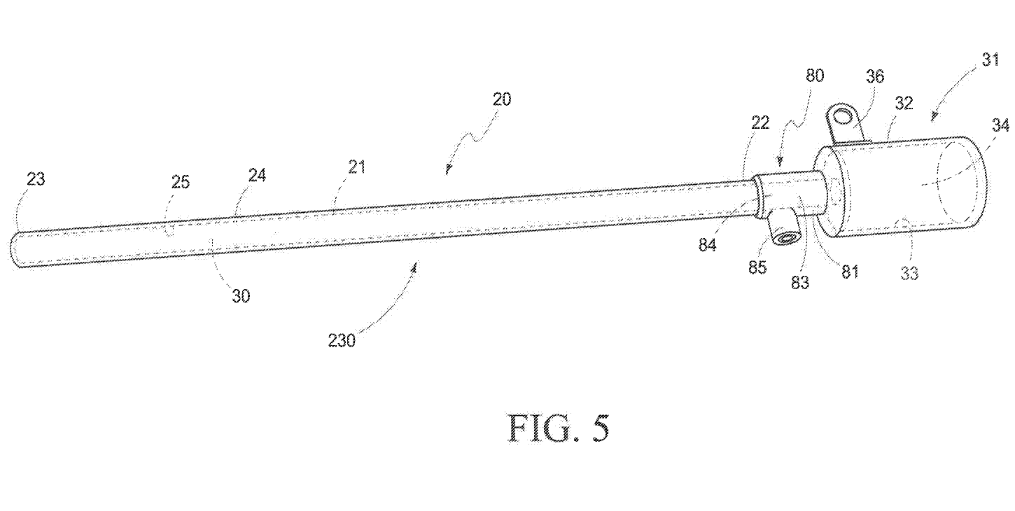

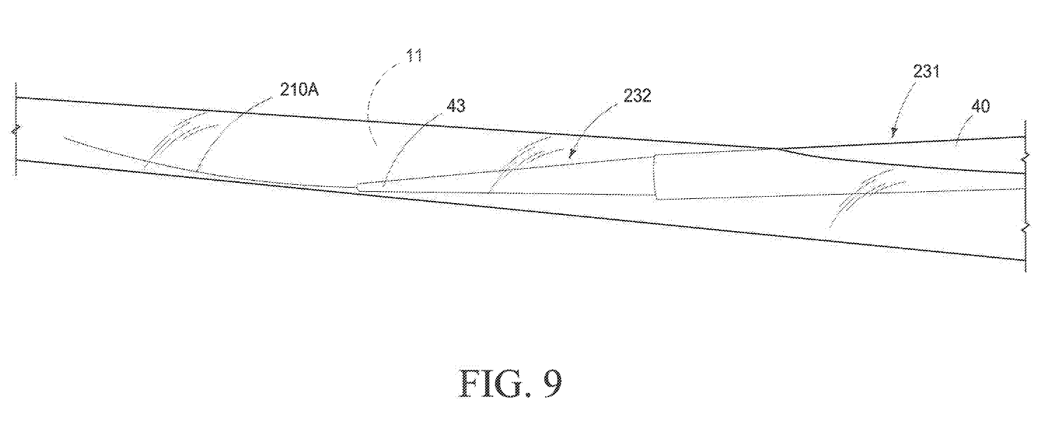

The methodology 200 of the present invention includes a sixth step 230 (FIG. 5) of providing a sheath 20 having opposite first, and second ends 42 and 43, and wherein the sheath further defines a longitudinally extending passageway 30 which has a predetermined inside diametral dimension. The method further includes a seventh step 231 which includes passing the wire, at least in part, through the longitudinally extending passageway 30 of the sheath 20 (FIG. 9); and an eighth step 232 of moveably advancing the sheath 20 along the wire. The methodology 200 further includes a ninth step 233 of inserting the second end 23 of the sheath 20 within the body cavity 11 which is being accessed (FIG. 10).

After the sixth step 230 of providing the sheath, the method 200 of the present invention further includes a tenth step 240 (FIG. 10), and which includes removing the wire from the body cavity 11 which is being accessed by forcibly withdrawing the wire through the longitudinally extending passageway 30 which is defined by the sheath 20. After the step of removing the wire from the body cavity 240 the method further includes an eleventh step 241 of providing an elongated dilator 40, having a main body 41, and which has an outside diametral dimension which is less than the inside diametral dimension of the longitudinally extending passageway 30 which is defined by the elongated sheath 20. The main body 41 of the elongated dilator 40 further has opposite first and second ends 42 and 43, and a longitudinally oriented lumen 50 which extends between the opposite first and second ends 42 and 43 of the main body 41. The main body 41 of the elongated dilator 40 further has an outside facing surface 44. Further, first and second passageways 51 and 52 are individually formed in the main body 41 of the elongated dilator 40. Each of the first and second passageways 51 and 52 have a proximal and a distal end 53 through 56, respectively. The respective first and second passageways 51 and 52 each extend from the first end 42 of the main body 41, and in the direction of the second or distal end 43. The method 200 further includes a twelfth step 242 of telescopingly inserting the main body 41 of the dilator 40 within the longitudinally extending passageway 30 which is defined by the sheath 20, and longitudinally advancing the dilator 40 along the longitudinally extending passageway 30 which is defined by the sheath 20; and a thirteenth step 243 of orientating the second end 43 of the dilator 40 at a location which is longitudinally outwardly relative to the second end 23 of the sheath 20, and within the body cavity 11 which is being accessed. The dilator 40 is selectively, reciprocally, and longitudinally moveable relative to the second end 43 of the elongated sheath 20.

The methodology of the present invention 200 includes a fourteenth step 250 of providing a selectively inflatable occlusion balloon 70, and mounting the occlusion balloon 70, on the dilator 40, and at a location 60 which is intermediate the first and second ends 42 and 43 thereof. The methodology includes a fifteenth step 251 of coupling the occlusion balloon 70 in selective fluid flowing relation relative to the distal end 54 of the first passageway 51, and which is formed in the main body 41 of the elongated dilator 40. The method includes a sixteenth step 251 of longitudinally advancing the occlusion balloon 70 relative to the dilator 40, and to a position where the occlusion balloon 70 is located longitudinally outwardly relative to the second end 23 of the elongated sheath 20. It should be understood that the second passageway 52 which is formed in the outside facing surface 44 of the main body 41 of the dilator 40 extends from the first end 42 thereof, and the distal or terminal end 56 of the second passageway 52 terminates at a location which is adjacent to, but just short of the selectively inflatable occlusion balloon 70. As seen in the drawings the main body 41 of the elongated dilator 40 is selectively, longitudinally advanced relative to the sheath 20 so as to position both the occlusion balloon 70, and the distal end of the passageway 52, and which is formed in the main body 41 of the elongated dilator 40, at a location which is longitudinally outwardly relative to the second end 23 of the sheath 20.

The methodology 200 of the present invention also includes a seventeenth step 260 of providing a first source of a pressurized fluid 101, and coupling the first source of pressurized fluid 101 in selective, fluid delivering relation relative to the proximal end 53 of the first passageway 51 of the elongated dilator 40. The method further includes an eighteenth step 261 of delivering the first source of pressurized fluid 100 to the proximal end of the first passageway 51 so as to effect an inflation 72, of the occlusion balloon 70, within the body cavity 11 which is being accessed. The methodology includes yet another nineteenth step 262 of providing a second source of pressurized fluid 102, and coupling the second source of pressurized fluid 102 in selective, fluid delivering relation relative to the proximal end 55 of the second passageway 52 of the elongated dilator 40. The method 200 further includes a twentieth step 263 of delivering the source of pressurized fluid 102 to the second passageway 52, and for movement therealong to the distal end 56 of the second passageway 52. The methodology includes another step of depositing 264 the second source of pressurized fluid 102 within the body cavity 11 being accessed (FIG. 11). It should be understood that the second source of pressurized fluid 102 exits the elongated dilator 40 in a direction which is oriented laterally, outwardly relative thereto, and into the body cavity 11 which is being accessed (FIG. 11).

### Operation

The operation of the described embodiments of the present invention are believed to be readily apparent and are briefly summarized at this point. In its broadest aspect the present device 10 for gaining access into a body cavity 11 includes an elongated sheath 20, having opposite ends 22 and 23, respectively, and which further defines a longitudinally oriented passageway 30 which extends between the opposite ends 22 and 23. The device 10 includes an elongated dilator 40 having a main body 41 with opposite ends, 42 and 43, respectively. The dilator 40 is telescopingly received within the longitudinally extending passageway 30, and which is defined by the sheath 20. The dilator 40 further defines a longitudinally oriented lumen 50 which extends between the opposite ends thereof 42 and 43. The elongated dilator further defines first and second passageways 51 and 52, and which are formed in the main body 41 of the elongated dilator 40. The device 10 of the present invention includes a selectively inflatable occlusion balloon 70 which is mounted on the dilator 40, and which further is coupled in fluid flowing relation relative to the first passageway 51, and which is defined by the elongated dilator 40. The device 10 also includes a first source of pressurized fluid 101, and which is coupled in fluid flowing relation relative to the first passageway 51. Finally the device 10 of the present invention includes a second source of pressurized fluid 102 which is coupled in fluid flowing relation relative to the second passageway 52. Upon insertion of at least a portion of the dilator 40 within the body cavity 11, and the further advancement of the main body 41 of the dilator 40 to a position where the occlusion balloon 70 is located in spaced relation relative to the second end 23 of the sheath 20, fluid supplied by the first source of fluid 101 can be used to inflate the first balloon to occlude a portion of the body cavity 11 for medical purposes which include effecting a reflux angiogram of an arterial anastomosis of a patient (FIG. 11). Still further, the present device 10 is designed so as to allow a clinician to selectively deliver a second source of fluid 102 into the body cavity 11 by supplying the second source of fluid 102 to the second passageway 52, and which then travels along to the terminal end 56 thereof, and then passes or escapes longitudinally outwardly, and into the body cavity 11 (FIG. 11). Again the second source of fluid 102 may include a flushing fluid or a contrast liquid which may enhance the production of x-rays of the blood vessels of the patient.

The present invention 10 also relates to a method 200 for gaining access into a body cavity of a patient and which includes, in its broadest aspect, the step 201 of selecting a body cavity 11 for access; and further inserting a needle 202 at least in part into the selected body cavity, and wherein the needle defines a longitudinally oriented passageway 205 extending therethrough. The method 200 includes another step 210 of inserting a wire into the longitudinally oriented passageway 205, and a step 211 of orienting the wire in a given location within the selected body cavity 11. The method includes another step of removing the needle 213 from the body cavity while simultaneously maintaining the wire within the selected body cavity. The methodology 200 also includes another step 230 of providing a sheath 20 having an elongated main body 21, and which defines a longitudinally extending passage 30 having a predetermined inside diametral dimension, and a step 231 of extending the wire through the longitudinally extending passageway 30. The present methodology includes another step 232 of moveably advancing the sheath 20 along the wire; and inserting, at least a portion of the sheath, into the selected body cavity 11 at a step 233. The methodology includes another step 240 of removing the wire from the selected body cavity while maintaining at least a portion of the sheath within the selected body cavity 11. The method includes another step 241 of providing a dilator with a main body 41, and which defines a longitudinally extending lumen 50, and first and second passageways 51 and 52, and telescopingly orienting the main body 41 of the dilator 40 within the longitudinally extending passageway 30 at step 242, which is defined by the sheath 20 and inserting in part a portion of the main body 41 of the dilator within the selected body cavity 11. This is seen at step 233. The methodology further includes another step 250 of mounting a selectively inflatable occlusion balloon 70 in a given location on the main body 41 of the dilator 40 and coupling the occlusion balloon 70 in fluid flowing relation relative to the first passageway 51 as defined by the dilator 40. The method includes another step 260 of selectively coupling a first source of pressurized fluid 101 to the first passageway 51, and which is defined by the dilator 40, and delivering 261 the first source of pressurized fluid 101 to the first passageway 51 so as to effect inflation 72 of the occlusion balloon 70 within the selected body cavity 11. The method includes another step 262 of selectively coupling a second source of a pressurized fluid 102 to the second passageway 52, and delivering 263 the second source of pressurized fluid 102 by way of the second passageway 52 to the selected body cavity 11.

Therefore it will be seen that the present invention provides a convenient device and method for gaining access into a body cavity and which avoids the shortcomings attendant with the prior art devices and methodology used heretofore and further allows a clinician a new means for removing blood from a dialysis patient and then returning the blood to the patient in an effective manner not possible with the techniques used before,

In compliance with the statute the invention has been described in language more or less specific as to structural and methodological features. It should be understood however that the invention is not limited to the specific features shown and described since the means herein disclosed comprise preferred forms of putting the invention into effect. The invention is therefore claimed in any of its forms or modifications within the proper scope of the appended claims appropriately interpreted in accordance with the Doctrine of Equivalence.

We claim:

1. A device for gaining access into a body cavity, comprising:
an elongated sheath having opposite ends, and further defining a longitudinally oriented passageway which extends between the opposite ends;
an elongated dilator having a main body with opposite ends, and which is telescopingly received within the longitudinally extending passageway which is defined by the sheath, and wherein the dilator defines a longitudinally oriented lumen which extends between the opposite ends thereof, and further has a first and second passageway which is formed in the main body of the elongated dilator;
a selectively inflatable occlusion balloon mounted on the dilator, and which is coupled in fluid flowing relation relative to the first passageway which is defined by the elongated dilator;
a first source of pressurized fluid which is coupled in fluid flowing relation relative to the first passageway; and
a second source of pressurized fluid which is coupled in fluid flowing relation relative to the second passageway.

2. A device as claimed in claim 1, and wherein the opposite ends of the sheath includes a proximal, first end, and a distal, second end, and wherein the sheath has an inside facing surface which defines the longitudinally oriented passageway, and wherein the longitudinally oriented passageway has a predetermined, and substantially uniform inside diametral dimension, and wherein the elongated sheath has an exterior facing surface, and the second passageway is defined, at least in part, by the exterior surface, and wherein the device further includes a fluid coupling which is mounted on the exterior facing surface of the sheath, and is further located at first end of the sheath, and which further fluid flowingly couples the first and second sources of pressurized fluids to each of the first and second passageways which are defined by the dilator.

3. A device as claimed in claim 2, and wherein the exterior facing surface of the elongated sheath is narrowly cylindrical, and wherein the fluid coupling is at least partially rotatable relative to the exterior facing surface of the sheath, and wherein the partial rotation of the fluid coupling relative to the sheath selectively couples each of the first and second sources of pressurized fluid in fluid flowing relation relative to the respective, first and second passageways which are defined by the main body of the dilator.

4. A device as claimed in claim 3, and wherein the opposite ends of the dilator includes a first end which is located in spaced relation relative to the first end of the sheath, and a second end which, when assembled with the sheath, is located in spaced relation relative to the second end of the sheath, and wherein the dilator has an outside diametral dimension which is less than the inside diametral dimension of the longitudinally oriented passageway as defined by the sheath, and wherein the dilator is selectively, reciprocally moveable relative to the sheath, and wherein the occlusion balloon is mounted at a location which is between the first and second ends of the dilator, and the second passageway which is defined by the main body of the dilator extends from the first end of the dilator, and terminates at a location which is in an adjacent, closely spaced relationship relative to the occlusion balloon, and wherein the dilator is longitudinally advanced relative to the sheath to a location where the occlusion balloon, and a terminal portion of the second passageway are each oriented longitudinally, outwardly, relative to the second end of the sheath.

5. A device as claimed in claim 4, and wherein the first source of the pressurized fluid is effective, when delivered to the first passageway, to inflate the occlusion balloon to a predetermined outside diametral dimension which at least partially occludes a body cavity of a patient.

6. A device as claimed in claim 4, and wherein the second source of pressurized fluid comprises a liquid which is delivered, at least in part, into a body cavity of a patient.

7. A device as claimed in claim 4, and wherein the sheath has an interior facing surface which defines the longitudinally extending passageway, and wherein the second passageway which is defined by the dilator is defined, at least in part, by the interior facing surface of the sheath.

8. A device as claimed in claim 4, and wherein the body cavity may include a fistula or other blood vessel, and wherein the occlusion balloon is inflated so as to effect a reflux angiogram of an arterial anastomosis of a patient.

9. A device as claimed in claim 4, and wherein the second end of the dilator, the occlusion balloon, and the terminal end of the second passageway, are each sized so as to be received within a body cavity of a patient.

10. A device as claimed in claim 4, and wherein the main body of the elongated dilator is flexible, and the second end of the dilator is stiff so as assist in the insertion of the second end of the dilator into a body cavity of a patient.

11. A device for gaining access into a body cavity, comprising:
an elongated sheath having opposite first, and second ends, an exterior facing surface, and a longitudinally extending passageway which extends between the opposite first and second ends, and wherein the longitudinally extending passageway has a predetermined inside diametral dimension;
an elongated dilator having a main body which has an outside diametral dimension which is less than the inside diametral dimension of the longitudinally extending passageway which is defined by the elongated sheath, and wherein the main body of the elongated dilator further has opposite, first and second ends, and wherein the second end of the dilator is received within a body cavity of a patient, and wherein the main body of the elongated dilator has a longitudinally oriented lumen which extends between the opposite first and second ends of the main body, and wherein the elongated dilator is further telescopingly received within the longitudinally extending passageway of the sheath, and is further reciprocally and longitudinally moveable relative to the elongated sheath, and wherein the main body of the elongated dilator further has an outside facing surface, and wherein a first and second passageway is individually formed in the main body of the elongated dilator, and which further, respectively, extend from the first end thereof, and in the direction of the second end of the main body, and wherein each of the first and second passageways have a proximal, and a distal end;
a selectively inflatable occlusion balloon mounted on the dilator at a location which is intermediate the first and second ends of the main body thereof, and wherein the dilator balloon is coupled in selective, fluid flowing relation relative to the first passageway which is formed in the main body of the elongated dilator, and wherein the occlusion balloon can be inflated when the elongated dilator is longitudinally advanced to a position where the occlusion balloon is located longitudinally, outwardly, relative to the second end of the elongated sheath, and wherein the second passageway which is formed in the main body of the dilator, and further defined, at least in part, by the exterior facing surface thereof, extends from the first end of the dilator in the direction of the second end of the dilator, and wherein the distal end of the second passageway terminates at a location which is adjacent to, but short of the selectively inflatable occlusion balloon, and wherein the main body of the elongated dilator can be longitudinally advanced relative to the sheath so as to position both the occlusion balloon, and the distal end of the second passageway which is formed in the main body of the elongated dilator, at a location which is longitudinally, outwardly relative to the second end of the sheath;
a first source of a pressurized fluid which is coupled in selective fluid delivering relation relative to the proximal end of the first passageway, and which is defined by the elongated dilator, and wherein the first source of pressurized fluid, when delivered to the first passageway is effective in inflating the occlusion balloon; and
a second source of a pressurized fluid which is coupled in selective, fluid delivering relation relative to the proximal end of the second passageway of the elongated dilator, and which further, when delivered to the second passageway, travels to the distal end thereof, and then exists the elongated dilator in a direction which is oriented laterally, outwardly relative thereto.

12. A device as claimed in claim 11, and wherein the elongated sheath has an interior facing surface, and the second passageway is defined, at least in part, by the interior facing surface of the elongated sheath, and wherein the device further includes a first fluid coupling portion which is mounted on the exterior facing surface of the sheath, and is further located at the first end of the sheath, and which further selectively fluid flowingly couples the second source of the pressurized fluid to the second passageway which is defined by the dilator.

13. A device as claimed in claim 12, and wherein, and wherein the dilator includes a second fluid coupling portion which is oriented in fluid delivering relation relative to the first passageway thereof, and wherein the first source of pressurized fluid is delivered to the first passageway so as to effect an inflation of the occlusion balloon.

14. A device as claimed in claim 13, and wherein the body cavity may include a fistula or other blood vessel, and wherein the occlusion balloon is inflated so as to effect a reflux angiogram of an arterial anastomosis of a patient.

15. A method for gaining access to a body cavity, comprising:
selecting a body cavity for access;
inserting a needle, at least in part, into the selected body cavity, and wherein the needle defines a longitudinally oriented passageway extending therethrough;
inserting a wire into the longitudinally oriented passageway, and orienting the wire in a given location within the selected body cavity;
removing the needle from the body cavity while maintaining the wire within the selected body cavity;
providing a sheath having a main body which defines a longitudinally extending passageway having a predetermined inside diametral dimension, and extending the wire through the longitudinally extending passageway;
movably advancing the sheath along the wire, and inserting at least a portion of the sheath into the selected body cavity;
removing the wire from the selected body cavity while maintaining at least a portion of the sheath within the selected body cavity;
providing a dilator with a main body which defines a longitudinally extending lumen, and first and second passageways, and telescopingly orienting the main body of the dilator within the longitudinally extending passageway which is defined by the sheath, and inserting, at least in part, a portion of the main body of the dilator within the selected body cavity;
mounting a selectively inflatable occlusion balloon in a given location on the main body of the dilator, and coupling the occlusion balloon in fluid flowing relation relative to the first passageway as defined by the dilator;
selectively coupling a first source of a pressurized fluid to the first passageway which is defined by the dilator, and delivering the first source of the pressurized fluid to the first passageway so as to effect an inflation of the occlusion balloon within the selected body cavity; and
selectively coupling a second source of a pressurized fluid to the second passageway, and delivering the second source of the pressurized fluid by way of the second passageway to the selected body cavity.

16. A method as claimed in claim 15, and wherein the sheath has opposite first and second ends, an inside facing surface which defines the longitudinally extending passageway, and an exterior facing surface, and wherein the dilator has opposite first and second ends, and an exterior facing surface, and wherein the second passageway is formed, at least in part, by the exterior facing surface of the dilator, and wherein the inside facing surface of the sheath forms, at least in part, a portion of the second passageway when the dilator is telescopingly oriented within the longitudinally extending passageway of the sheath.

17. A method as claimed in claim 16, and wherein the step of mounting the selectively inflatable balloon takes place at a location which is between the first and second ends of the main body of the dilator, and wherein the second passageway has a terminal end which is positioned adjacent to the occlusion balloon.

18. A method as claimed in claim 17, and which further comprises:
after the step of mounting a selectively inflatable occlusion balloon on the main body of the dilator, and before the step of selectively coupling the first source of pressurized fluid to the first passageway, movably advancing the main body of the dilator relative to the sheath so as to locate the occlusion balloon longitudinally outwardly relative to the second end of the sheath.

19. A method as claimed in claim 17, and which further comprises:
before the step of selectively coupling the second source of the pressurized fluid to the second passageway, movably advancing the main body of the dilator relative to the sheath so as to locate the terminal end of the second passageway longitudinally outwardly relative to the second end of the sheath.

20. A method for gaining access to the body cavity of a patient, comprising:
inserting an elongated needle, at least in part, into a body cavity of a patient, and wherein the elongated needle has a proximal and distal end, and further has a longitudinally oriented passageway which extends between the proximal and distal ends thereof;
inserting a wire into the longitudinally extending passageway of the elongated needle, and advancing the inserted wire through the needle, and further orienting a portion of the wire in the body cavity which is being accessed;
after the step of inserting a portion of the wire into the body cavity which is being accessed, removing the needle from the body cavity by passing the needle over the wire, and simultaneously leaving the portion of the wire previously inserted in the body cavity which is being accessed, within the body cavity;
providing a sheath having opposite first, and second ends, and a longitudinally extending passageway which extends between the opposite first and second ends, and wherein the longitudinally extending passageway of the sheath has a predetermined inside diametral dimension, and wherein the method further includes passing the wire, at least in part, through the longitudinally extending passageway of the sheath, and further moveably advancing the sheath along the wire, and inserting the second end of the sheath within the body cavity which is being accessed;
after the step of providing the sheath, the method includes another step of removing the wire from the body cavity which is being accessed by forcibly withdrawing the wire through the longitudinally extending passageway which is defined by the sheath;
after the step of removing the wire from the body cavity, the method further includes a step of providing an elongated dilator having a main body which has an outside diametral dimension which is less than the inside diametral dimension of the longitudinally extending passageway which is defined by the elongated sheath, and wherein the main body of the elongated dilator further has opposite, first and second ends, and a longitudinally oriented lumen which extends between the opposite first and second ends of the main body, and wherein the main body of the elongated dilator further has an outside facing surface, and wherein a first and second passageway is individually formed in the main body of the elongated dilator, and wherein each of the first and second passageways have a proximal, and a distal end, and which further, respectively, extend from the first end thereof, and in the direction of the second end of the main body, and wherein the method further includes the step of telescopingly inserting the main body of the dilator within the longitudinally extending passageway which is defined by the sheath, and longitudinally advancing the dilator along the longitudinally extending passageway which is defined by the sheath, and orienting the second end of the dilator at a location which is longitudinally, outwardly relative to the second end of the sheath, and within the body cavity being accessed, and wherein the dilator is selectively, reciprocally, and longitudinally moveable relative to the second end of the elongated sheath;
providing a selectively inflatable occlusion balloon, and mounting the occlusion balloon on the dilator at a location which is intermediate the first and second ends of the main body thereof, and further coupling the dilator balloon in selective, fluid flowing relation relative to the distal end of the first passageway which is formed in the main body of the elongated dilator, and wherein the method further includes longitudinally advancing the occlusion balloon relative to the dilator to a position where the occlusion balloon is located longitudinally, outwardly, relative to the second end of the elongated sheath, and wherein the second passageway which is formed in the outside facing surface of the main body of the dilator extends from the first end thereof, and the distal end of the second passageway terminates at a location which is adjacent to, but short of the selectively inflatable occlusion balloon, and wherein the main body of the elongated dilator is selectively, longitudinally advanced relative to the sheath so as to position both the occlusion balloon, and the distal end of the second passageway, and which is formed in the main body of the elongated dilator, at a location which is longitudinally, outwardly relative to the second end of the sheath;
providing a first source of a pressurized fluid and coupling the first source of pressurized fluid in selective, fluid delivering relation relative to the proximal end of the first passageway of the elongated dilator, and delivering the first source of the pressurized fluid to the proximal end of the first passageway so as to effect an inflation of the occlusion balloon within the body cavity which is being accessed; and
providing a second source of a pressurized fluid, and coupling the second source of the pressurized fluid in selective, fluid delivering relation relative to the proximal end of the second passageway of the elongated dilator, and wherein the method further includes the step of delivering the second source of pressurized fluid to the second passageway, for movement therealong to the distal end of the second passageway, and further depositing the second source of the pressurized fluid within the body cavity being accessed, and wherein the second source of pressurized fluid exists the elongated dilator in a direction which is oriented laterally, outwardly relative thereto, and into the body cavity being accessed.
